(19) United States
(12) Patent Application Publication
(10) Pub. No.: US 2003/0147771 A1
(43) Pub. Date: Aug. 7, 2003

(54) Waste material sterilizing and size reduction apparatus

(76) Inventor: Gerard Hodgins, Drogheda (IE)

Correspondence Address: John W. Hayes Esquire, John W Hayes PC, P.O. Box 365, Arlington Height, IL, 60006-0365, US

(21) Appl. No.: 10/071,501

(22) Filed: Feb. 7, 2002

(51) Int. Cl. A61L 2/08; A61L 2/00; A61L 9/00

(52) U.S. Cl. 422/26; 422/1; 422/38; 422/295; 422/298; 422/300; 422/305; 422/307

(57) ABSTRACT

A Waste Material Sterilizing and Size Reduction Apparatus A waste treatment apparatus and a method of treating waste using such an apparatus is described. The apparatus comprises a sealable vessel with an auger mounted therein. The vessel is adapted to receive the waste, and is then sealed. The application of steam to the waste which is rotated within the vessel using the auger effects a sterilizing of the waste material.

### FIELD OF THE INVENTION

The invention relates to the disposal of waste and particularly to a unit adapted to sterilize and volume reduce waste material.

### BACKGROUND TO THE INVENTION

The treatment and disposal of waste material presents many problems. Traditionally land fill sites have been used wherein a large area of land is used to house uncompacted waste material which is deposited there. Over a period of time the site becomes more and more full and it is necessary to close a specific site and open alternative sites. The use of land fill sites has attracted recent opposition in that the large areas required for such sites can present eyesores, as the waste is usually untreated can present hygiene problems and furthermore large tracts of lands become unusable for other purposes such as housing etc.

Alternative suggestions to waste disposal include large scale incinerators. These systems operate on the principal of burning the waste at high temperatures so as to eliminate much of the bulk of the waste. Although these are less demanding on the area of land required for the facilities, there are disadvantages in that there are perceptions that the incinerating of waste can present air-borne allergens and also there is a large capital investment required.

It is also well known in the treatment of medical and biological waste to use high temperature steam in a pressurised environment to sterilize the waste into a more manageable product. The use of such autoclaves is described in EP 1010399, GB 2152378, DE19600706, U.S. 5,091,158 and U.S. Pat. No. 5,424,033. These arrangements are specific to the treatment of medical waste and include modifications to treat the condensate resulting from the autoclave process and pre-shredding of the waste prior to the treatment within the autoclave.

Problems with these known systems is that the waste material usually can only be presented from the front of the devices, thereby limiting the volume of material that can be introduced. Additionally the entry port to the pressure vessel in typically also the exit port for the removal of the treated waste, which introduces problems with removal of material once treated. Accordingly there is a requirement for an improved waste system for treating and volume reducing waste material into a product suitable for disposal into a sanitary landfill site.

### OBJECT OF THE INVENTION

It is an object of the present invention to provide an improved waste treatment apparatus.

### SUMMARY OF THE INVENTION

Accordingly the present invention provides a waste sterilization and size reduction apparatus comprising:

a sealable pressurisable treatment vessel having an internally mountable auger, the mounted auger adapted to rotate relative to a longitudinal axis of the sealable vessel, the vessel having an entry port and an exit port, the entry port adapted to effect the introduction of waste material onto the auger, and the exit port adapted to effect the removal of waste material once treated within the vessel from the vessel, the vessel being further adapted to incorporate a plurality of high temperature steam entry ports, and wherein on introduction of waste material into the vessel and the sealing of the vessel, the agitation of the waste material within the vessel effected by the rotation of the auger and the concurrent passage of high temperature steam under pressure onto the agitated material affects a sterilization and size reduction of the waste material.

The apparatus is preferably adapted for the treatment of municipal waste.

The steam entry ports are preferably located along an axis of the auger so as to effect the introduction of steam outwardly onto the waste material disposed therein or arranged on an outside surface of the treatment vessel so as to effect the introduction of steam inwardly onto the waste contained within the treatment vessel.

The invention also provides a method of treating waste materials comprising the steps of:

introducing waste material through an entry door onto an auger mounted within a sealable vessel,

sealing the vessel,

effecting the creation of a vacuum within the vessel,

applying steam to the waste material contained within the vessel and agitating the waste material by a rotation of the auger in both a forward and reverse direction while the steam is being applied, and wherein during the application of the steam to the waste material the temperature within the vessel is maintained above 120° C.

The temperature is preferably at least 130° C. and more preferably at least 134° C.

The steam is applied for a time period typically about 40 minutes.

On completion of the cycle the treated waste material is removed from the sealable vessel via an exit door remotely located from the entry door.

Other aspects of the invention will be better understood with reference to the accompanying drawings.

### BRIEF DESCRIPTION OF THE DRAWINGS

FIG. 1 is a view from the side of a treatment apparatus according to the present invention,

FIG. 2 is a detailed view from the side of an auger according to a second embodiment of the present invention, and

FIG. 3 is a view along the axis of the auger of FIG. 2.

### DETAILED DESCRIPTION OF THE DRAWINGS

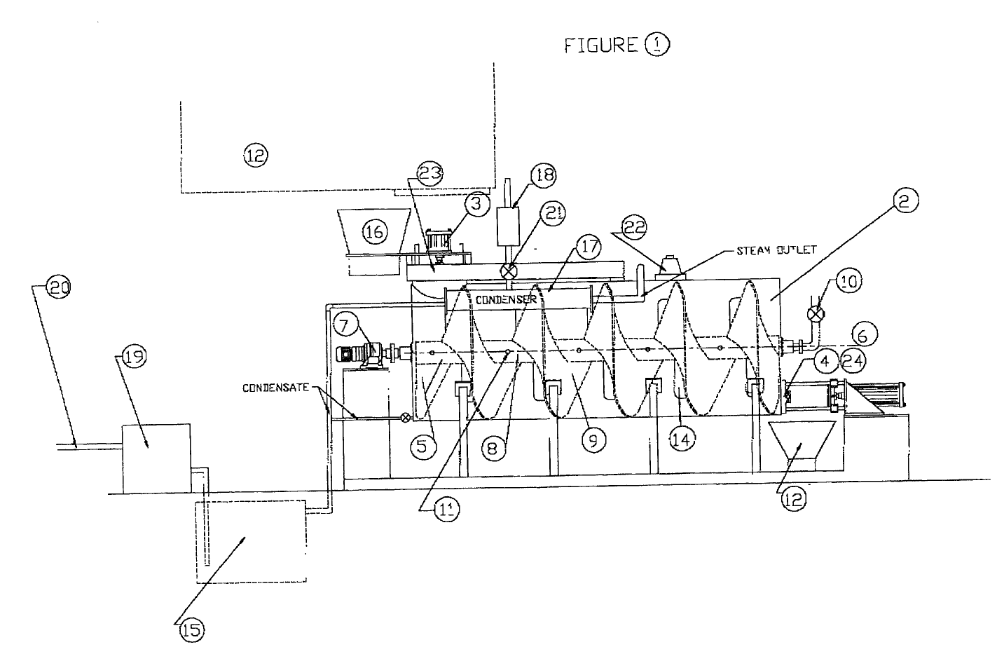

FIG. 1 is a view from the side of a treatment apparatus 1 according to the present invention. The apparatus 1 comprises a sealable treatment vessel 2 having an entry port 3 and an exit port 4. An internally mountable auger 5 extends along a longitudinal axis 6 of the vessel 2. Rotation of the auger within the vessel is effected by a conventional gearing mechanism 7 located outside the vessel. The auger 5 comprises an axle 8 and a screw thread 9 extending from the axle 8. The diameter of the thread 9 is preferably such that the cross-sectional area of the auger substantially equates to the inner diameter of the vessel. The thread is desirably also provided with lifting fins 14 which assist in an agitation of the waste during the process.

The entry 3 and exit 4 ports are provided with sealable doors 23, 24 which when the apparatus is in use are closed so as to effect a sealing of the vessel. The pressure within the vessel may be altered using a pressure control valve 10 so as to effect an increasing of the pressure within the vessel. A plurality of steam entry ports 11, to effect the introduction of steam into the vessel, are provided in this embodiment along the axle 8 of the auger 5. In an alternative embodiment, not shown, external manifolds may be utilised to effect the introduction of steam through the side walls of the vessel 2.

The filling of the vessel is achieved using an overhead hopper 12, having a hopper funnel 16, which is adapted to dispense waste material through the entry port 3 and onto the auger 5. The rotation of the auger 5 in the filling process facilitates the passage of waste material into the vessel. The filling process is typically continued until the vessel is approximately 70-90% and preferably 80-85% full. The vessel is filled to approximately 80% full to enable the auger to rotate in a forward and reverse direction without compressing the waste material within. If completely full this could not be achieved without causing too much pressure on the drive mechanism and would also effect a compressing of the waste which is not a function of the system of the present invention.

Once filled the hopper funnel 16 is moved away from the entry port which is closed and the pressure within the vessel increased to approximately 45-50 psi.(approximately 3 atmospheres pressure). This is achieved through the pressure of the steam itself.

A vacuum is created prior to the introduction of the steam and is used to remove the air from with the chamber vessel 2 so as to remove air pockets which would inhibit the function of the steam by creating cold spots.

This vacuum is achieved by injecting steam into an external condenser 17 and opening a vessel exhaust valve 21 thus sucking out the air from within via a carbon (or alternative type) filter 18.

As such the process can be divided into two stages: the creation of the vacuum is stage 1 of the process and the introduction of the steam is stage 2.

Steam at approximately 150° C. is introduced from the boiler through pressure control valve 10 to the steam entry ports and the waste contained within the vessel is agitated by means of rotation of the auger in both directions for a predetermined time period, typically not less than 8 minutes and desirably about 40 minutes. As a result of the agitation all the waste within the vessel comes into contact with the applied steam. During the process the temperature within the vessel desirably does not drop below 134° C.

The condensate is removed through the condenser 17 and a valve at the base of the vessel to a holding tank 15 and is then filtered at a filtration unit 19 and returns via piping 20 to the boiler ( not shown). Typically the boiler requires an output of approximately 10 tonnes per hour gas burner/ steam boiler.

An emergency pressure release valve 22 may also be provided in case of situations where immediate reduction of pressure from within the vessel is required.

Once the treatment process is completed the steam contained within the vessel is removed from the vessel to the condenser. Drying of the material contained within the vessel is effected once the steam has been removed from the vessel. The drying results from the heat within the vessel generating more steam from the waste material which is then drawn out of the vessel via the condenser as described previously.

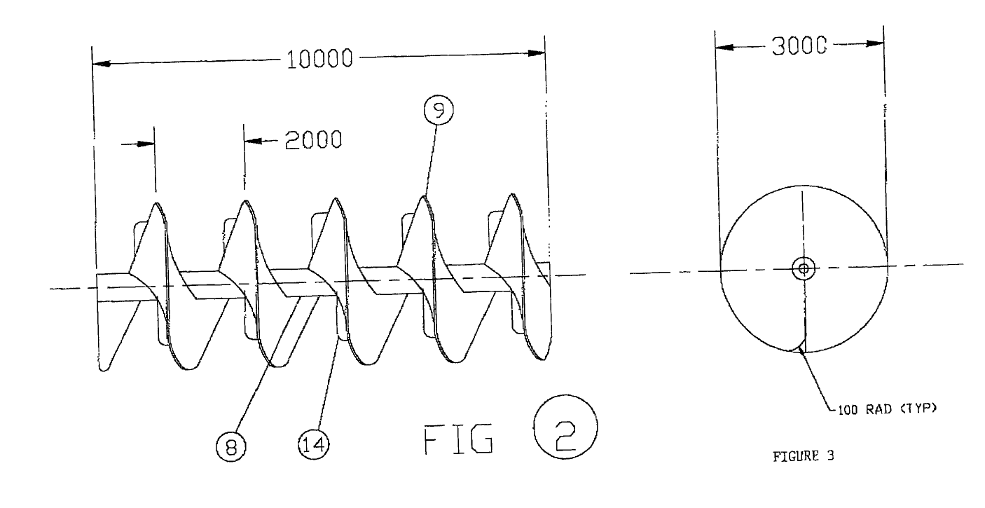

FIG. 2 shows an alternative embodiment of an auger. The same reference numerals are used for similar components of the auger. FIG. 2 also illustrates typical dimensions of the auger in mm. It will be seen that the auger is typically 10 m long and has a total diameter of 3 m. Typical distances between adjacent threads is 2 m. The fins are typically offset at 100 rad from an axis of the auger. In this embodiment the steam entry ports are not provided on the axis of the auger. In such situations the vessel will have to be adapted to allow the steam to enter the vessel through side walls or outer surface of the vessel via manifolds (not shown).

FIG. 3 shows a view along the axis of the auger of FIG. 2 and details a typical diameter for the auger of approximately 3 m.

Typical dimensions of the apparatus of the present invention would allow approximately 10 tonnes of waste material to be treated at any one time. For domestic waste this typically requires a treatment vessel having a longitudinal length of 10 m and a diameter of 3 m. The hopper used is dimensioned to contain up to 25 tonnes of material and the complete processing of 10 tonnes of material could be effected within a one hour time period. By treating the waste material at high temperatures and pressures it is possible to reduce the waste volume to approximately ⅛th its original dimensions. The size reduction is achieved by a combination of heat and pressure and the removal of moisture from the waste in drying cycle at the end of process. Just as in pressure cooking (without the drying cycle) the items being cooked for long enough will reduce. By application of this technique to paper, cardboard, food and even plastic containers, the results are dramatic in size reduction, although it will be appreciated that large amounts of solids will inhibit the amount of size reduction achievable. The sterilized material is then removed from the exit port 4, the forward rotation of the auger removes the waste from the vessel via the exit port to the output conveyor 12 as shown, into a dump truck (not shown) wherein it may be transported to a landfill site as sterile waste.

Although described with reference to domestic waste it will be appreciated by those skilled in the art that the apparatus of the present invention may also be adapted to allow the treatment of medical waste. There would be modifications required regarding control and confirmation of sterilisation and also of environmental conditions, i.e. hopper and surrounding area. The apparatus of the present invention offers many advantages over the systems of the prior art including:

bulk handling,

greater throughput,

larger volume per cycle

limited emission,

self cleaning and

inexpensive to install and operate.

The words “comprises/comprising” and the words “having/including” when used herein with reference to the present invention are used to specify the presence of stated features, integers, steps or components but does not preclude the presence or addition of one or more other features, integers, steps, components or groups thereof.

1. A waste sterilization and size reduction apparatus comprising:
a sealable pressurisable treatment vessel having an internally mountable auger, the mounted auger adapted to rotate relative to a longitudinal axis of the sealable vessel, the vessel having an entry port and an exit port, the entry port adapted to effect the introduction of waste material onto the auger, and the exit port adapted to effect the removal of waste material once treated within the vessel from the vessel, the vessel being further adapted to incorporate a plurality of high temperature steam entry ports, and wherein on introduction of waste material into the vessel and the sealing of the vessel, the agitation of the waste material within the vessel effected by the rotation of the auger and the concurrent passage of high temperature steam under pressure onto the agitated material affects a sterilization and size reduction of the waste material.

2. The apparatus as claimed in claim 1 wherein the waste material is municipal waste.

3. The apparatus as claimed in claim 1 or claim 2 wherein the steam entry ports are located along an axis of the auger so as to effect the introduction of steam outwardly onto the waste material disposed therein.

4. The apparatus as claimed in claim 1 or claim 2 wherein the steam entry ports are arranged on an outside surface of the treatment vessel so as to effect the introduction of steam inwardly onto the waste contained within the treatment vessel.

5. The apparatus as claimed in any preceding claim wherein the auger comprises an axle and a screw thread extending from the axle, the diameter of the screw thread being such that the cross-sectional area of the auger substantially equates to the inner diameter of the vessel.

6. The apparatus as claimed in any preceding claim further comprising lifting fins adapted to assist in an agitation of the waste.

7. The apparatus as claimed in any preceding claim wherein the entry and exit ports are provided with sealable doors which when the apparatus is in use are closed to effect a sealing of the vessel.

8. The apparatus as claimed in any preceding claim further comprising a pressure control valve adapted to effect an altering of the pressure within the vessel.

9. The apparatus as claimed in any preceding claim further comprising a hopper adapted to dispense waste through the entry port and onto the auger.

10. The apparatus as claimed in any preceding claim wherein the auger is about 10 m long.

11. A method of treating municipal waste materials comprising the steps of:
a) introducing waste material through an entry door onto an auger mounted within a sealable vessel,
b) sealing the vessel,
c) effecting the creation of a vacuum within the vessel,
d) applying steam to the waste material contained within the vessel and agitating the waste material by a rotation of the auger in both a forward and reverse direction while the steam is being applied, and
wherein during the application of the steam to the waste material the temperature within the vessel is maintained above 120° C.

12. The method as claimed in claim 11 wherein the temperature is preferably at least 130° C. and more preferably at least 134° C.

13. The method as claimed in claim 11 or 12 wherein the steam is applied for a time period of about 40 minutes.

14. The method as claimed in any one of claims 11 to 13 wherein on completion of the cycle the treated waste material is removed from the sealable vessel via an exit door remotely located from the entry door.

15. The method as claimed in any one of claims 11 to 14 wherein the vacuum is created within the vessel by injecting steam into an external condenser and opening a vessel exhaust valve, thereby drawing air out from the vessel.

16. The method as claimed in any one of claims 11 to 15 wherein any condensate formed during the application of steam to the waste material is removed from the vessel.

17. The method as claimed in claim 16 wherein the withdrawn condensate is filtered prior to being returned to a boiler for the formation of additional steam to be introduced to the vessel.

18. The method as claimed in any one of claims 11 to 17 wherein on completion of the treatment process any remaining condensate is removed from the vessel and the material contained within the vessel is dried.

19. A method for the treatment of waste materials substantially as hereinbefore described with reference to FIG. 1 or FIGS. 2 and 3 of the accompanying drawings.

20. A waste treatment apparatus for the treatment of waste materials substantially as hereinbefore described with reference to FIG. 1 or FIGS. 2 and 3 of the accompanying drawings.
